(19) United States
(12) Patent Application Publication
(10) Pub. No.: US 2026/0044805 A1
(43) Pub. Date: Feb. 12, 2026

(54) METHOD AND APPARATUS FOR ASSESSING WORK PERFORMANCE OF TARGET ORGANIZATION, AND ELECTRONIC DEVICE

(71) Applicant: National Center for Chronic and Noncommunicable Disease Control and Prevention, Chinese Center for D, Beijing (CN)

(72) Inventor: Lei Hou, Beijing (CN)

(21) Appl. No.: 19/365,152

(22) Filed: Oct. 21, 2025

Related U.S. Application Data
(63) Continuation of application No. PCT/CN2024/083344, filed on Mar. 22, 2024.

(30) Foreign Application Priority Data
Apr. 21, 2023 (CN) 202310436219.5

(51) Int. Cl. G06Q 10/0639; G16H 40/20

(52) U.S. Cl. CPC G06Q 10/06393; G16H 40/20

(57) ABSTRACT

A method for evaluating work performance of a target organization includes: acquiring at least one set of witness information data pairs within a business coverage area of the target organization and within a preset historical time period, each set of witness information data pairs including a witness rate and a witness action rate; calculating a rescue action conversion efficiency corresponding to the target organization based on the at least one set of witness information data pairs; and evaluating the work performance of the target organization based on the rescue action conversion efficiency corresponding to the target organization.

### CROSS-REFERENCE TO RELATED APPLICATIONS

This application is a continuation of PCT/CN2024/083344, filed on Mar. 22, 2024 and claims priority of Chinese Patent Application No. 202310436219.5, filed on Apr. 21, 2023, the entire contents of which are incorporated herein by reference.

### TECHNICAL FIELD

The disclosure relates to the field of computer technologies, and more particularly to a method and apparatus for assessing work performance of a target organization, and an electronic device.

### BACKGROUND

Out-of-hospital cardiac arrest (OHCA) is the most critical clinical condition, which mostly occurs in general population outside the hospital and lacks effective preventive measures. Its rescue follows the chain of survival principle, namely early recognition, early cardiopulmonary resuscitation (CPR), early defibrillation, early advanced life support, comprehensive treatment, and rehabilitation. Since it is difficult for emergency medical services (EMS) to arrive within the golden hour (generally 4 minutes, and the average EMS response time in China is approximately 15 minutes) after the incident occurs, the presence of someone (often non-professionals) at the scene to perform CPR is the only hope for survival from OHCA patients. Currently, the discharge survival rate for OHCA patients in China ranges between 0% and 0.61%. Fewer than half of OHCA patients are witnessed, with only 4.8% of patients receiving bystander CPR and the rate of bystander defibrillation (using an automated external defibrillator, i.e., AED) being nearly zero.

Since 1990, the international community has adopted the standardized Utstein reporting template to record the rescue process. Both the chain of survival and the Utstein reporting template describe and record the rescue process centered on OHCA patients. However, after cardiac arrest occurs, the patients lose consciousness and subjective initiative, and can only passively accept observation and treatment. Whether the patients receive treatment and whether their life can be sustained depends entirely on whether there are witnesses at the scene and whether those witnesses take rescue actions. These two aspects are related to the work of medical rescue-related organizations (e.g., medical rescue training organizations, medical rescue organizations, etc.). However, current research centered on non-patient bystanders is quite scarce, and for medical rescue-related organizations, it is also difficult to effectively assess their work performance based on existing assessment criteria.

Therefore, how to effectively assess medical rescue-related organizations is a technical problem that urgently needs to be solved.

### SUMMARY

Embodiments of the disclosure provide a method for assessing work performance of a target organization, an apparatus for assessing work performance of a target organization, and an electronic device, which are used to effectively assess medical rescue-related organizations.

In an embodiment of the disclosure, a method for evaluating work performance of a target organization is provided. The method includes: acquiring at least one set of witness information data pairs within a business coverage area of the target organization and within a preset historical time period; where each set of the witness information data pairs includes a witness rate and a witness action rate, and corresponds to a disease process state of target patients, the witness rate is a percentage of target patients in a certain disease process state being witnessed, the witness action rate is a proportion of witnesses, among all witnesses, who take preset rescue measures upon discovering the target patients in a certain disease process state, and the witnesses belong to a target population; calculating a rescue action conversion efficiency corresponding to the target organization based on the at least one set of witness information data pairs, where the rescue action conversion efficiency is a proportion, among all target patients in any disease process state, of target patients who receive effective preset rescue measures from members of the target population; and evaluating the work performance of the target organization based on the rescue action conversion efficiency corresponding to the target organization.

In some embodiments, the witness rate is obtained by: obtaining the witness rate based on the number of target patients witnessed while in a certain disease process state and the total number of target patients in the certain disease process state.

In some embodiments, the target patient is an out-of-hospital cardiac arrest patient; the disease process state is a cardiac arrest onset state, and the obtaining the witness rate based on the number of target patients witnessed while in a certain disease process state and the total number of target patients in the certain disease process state includes: obtaining a first witness rate based on a ratio between the number of out-of-hospital cardiac arrest patients witnessed while in the cardiac arrest onset state and the total number of out-of-hospital cardiac arrest patients in the cardiac arrest onset state; or the disease process state is a post-cardiac arrest unresponsive state, and the obtaining the witness rate based on the number of target patients witnessed while in a certain disease process state and the total number of target patients in the certain disease process state included: obtaining a second witness rate based on a ratio between the number of out-of-hospital cardiac arrest patients witnessed while already in the post-cardiac arrest unresponsive state and the total number of out-of-hospital cardiac arrest patients in the post-cardiac arrest unresponsive state.

In some embodiments, the witness action rate is obtained by: obtaining the witness action rate based on the number of witnesses who take preset rescue measures when the target patient is in a certain disease process state and the total number of witnesses.

In some embodiments, the target patient is an out-of-hospital cardiac arrest patient; the disease process state is a cardiac arrest onset state, and the obtaining the witness action rate based on the number of witnesses who take preset rescue measures when the target patient is in a certain disease process state and the total number of witnesses includes: obtaining a first witness action rate based on a ratio between the number of witnesses who take preset rescue measures when the out-of-hospital cardiac arrest patient is in the cardiac arrest onset state and the total number of witnesses who witness the out-of-hospital cardiac arrest patient in the cardiac arrest onset state; or the disease process state is a post-cardiac arrest unresponsive state, and the obtaining the witness action rate based on the number of witnesses who take preset rescue measures when the target patient is in a certain disease process state and the total number of witnesses includes: obtaining a second witness action rate based on a ratio between the number of witnesses who take preset rescue measures when the out-of-hospital cardiac arrest patient is in the post-cardiac arrest unresponsive state and the total number of witnesses who witness the out-of-hospital cardiac arrest patient in the post-cardiac arrest unresponsive state; and the preset rescue measures include one or more combinations of: manual cardiopulmonary resuscitation and use of an automated external defibrillator.

In some embodiments, the calculating the rescue action conversion efficiency corresponding to the target organization based on the at least one set of witness information data pairs includes: for each set of the witness information data pairs, calculating a product of the witness rate and the witness action rate in the set of the witness information data pairs; and obtaining the rescue action conversion efficiency based on the products of the witness rates and the witness action rates from all sets of the witness information data pairs.

In some embodiments, the rescue action conversion efficiency is obtained by the following calculation formula:

$$T = {\sum }_{i = 1}^{n} {k}_{i} {w}_{i} * {t}_{i}$$

where T is the rescue action conversion efficiency, wᵢ is the witness rate in the i-th set of witness information data pairs, tᵢ is the witness action rate in the i-th set of witness information data pairs, n is the number of the at least one set of witness information data pairs, and kᵢ is an effect coefficient corresponding to the i-th set of witness information data pairs, the effect coefficient is used to represent the effect of being administered the preset rescue measures when the target patient is in a certain disease process state.

In some embodiments, the target patient is an out-of-hospital cardiac arrest patient; the at least one set of witness information data pairs includes: a first witness information data pair and a second witness information data pair; the first witness information data pair corresponds to the cardiac arrest onset state of the out-of-hospital cardiac arrest patient; the second witness information data pair corresponds to the post-cardiac arrest unresponsive state of the out-of-hospital cardiac arrest patient; and the rescue action conversion efficiency corresponding to the target organization is calculated by the following formula:

$$T = {w}_{1} * {t}_{1} + k {w}_{2} * {t}_{2}$$

where T is the rescue action conversion efficiency corresponding to the target organization, w₁ is the witness rate in the first witness information data pair, t₁ is the witness action rate in the first witness information data pair, w₂ is the witness rate in the second witness information data pair, t₂ is the witness action rate in the second witness information data pair, and k is an effect coefficient determined based on a ratio between the rescue effect when the out-of-hospital cardiac arrest patient is in the post-cardiac arrest unresponsive state and the rescue effect when the out-of-hospital cardiac arrest patient is in the cardiac arrest onset state.

In some embodiments, the assessing the work performance of the target organization based on the rescue action conversion efficiency corresponding to the target organization includes: constructing a frequency table of rescue action conversion efficiencies (also referred to as rescue action conversion efficiency frequency table) based on multiple rescue action conversion efficiencies including the rescue action conversion efficiency corresponding to the target organization; each rescue action conversion efficiency in the multiple rescue action conversion efficiencies corresponds to one organization; obtaining a percentile corresponding to the target organization based on the frequency table of rescue action conversion efficiencies; and obtaining a work performance score of the target organization based on the percentile.

In an embodiment of the disclosure, a method for evaluating work performance of a target organization is provided. The method includes: acquiring, within a business coverage area of the target organization and a preset historical time period, at least one set of trainee witness information data pairs; where each set of the trainee witness information data pairs includes a trainee witness rate and a trainee action rate, and corresponds to a disease process state of a target patient; the trainee witness rate is a proportion, among all trainees trained by the target organization, of trainees who witness a target patient in a certain disease process state; the trainee witness action rate is a proportion, among all trainees trained by the target organization, of trainees trained by the target organization who take preset rescue measures upon discovering the target patient in a certain disease process state; calculating a work performance score of the target organization based on the at least one set of trainee witness information data pairs; and assessing the work performance of the target organization based on the work performance score of the target organization.

In some embodiments, the target patient is an out-of-hospital cardiac arrest patient; the disease process state is a cardiac arrest onset state, and the trainee witness rate is obtained by: obtaining a first trainee witness rate based on a ratio between the number of trainees, among those trained by the target organization, who witness a target patient in the cardiac arrest onset state and the total number of trainees trained by the target organization; or the disease process state is a post-cardiac arrest unresponsive state, and the trainee witness rate is obtained by: obtaining a second trainee witness rate based on a ratio between the number of trainees, among those trained by the target organization, who witness a target patient in the post-cardiac arrest unresponsive state and the total number of trainees trained by the target organization.

In some embodiments, the target patient is an out-of-hospital cardiac arrest patient; the disease process state is a cardiac arrest onset state, and the trainee action rate is obtained by: obtaining a first trainee action rate based on a ratio between the number of trainees trained by the target organization who take preset rescue measures upon discovering the target patient in the cardiac arrest onset state and the total number of all trainees trained by the target organization; or the disease process state is a post-cardiac arrest unresponsive state, and the trainee action rate is obtained by: obtaining a second trainee action rate based on a ratio between the number of trainees trained by the target organization who take preset rescue measures upon discovering the target patient in the post-cardiac arrest unresponsive state and the total number of all trainees trained by the target organization; and the preset rescue measures include one or more combinations of: manual cardiopulmonary resuscitation and use of an automated external defibrillator.

In some embodiments, the calculating the work performance score of the target organization based on the at least one set of trainee witness information data pairs includes: for each set of the trainee witness information data pairs, calculating a product of the trainee witness rate and the trainee action rate in that set of trainee witness information data pairs; and obtaining the work performance score of the target organization based on the products of the trainee witness rates and the trainee action rates from all sets of trainee witness information data pairs.

In some embodiments, the target patient is an out-of-hospital cardiac arrest patient; the at least one set of trainee witness information data pairs includes: a first trainee witness information data pair and a second trainee witness information data pair; the first trainee witness information data pair corresponds to the cardiac arrest onset state; and the second trainee witness information data pair corresponds to the post-cardiac arrest unresponsive state; the calculation formula for the work performance score of the target organization is as follows:

$$F = {a}_{1} * {b}_{1} + k {a}_{2} * {b}_{2}$$

where F is the work performance score of the target organization, a₁ is the trainee witness rate in the first trainee witness information data pair, b₁ is the trainee action rate in the first trainee witness information data pair, a₂ is the trainee witness rate in the second trainee witness information data pair, b₂ is the trainee action rate in the second trainee witness information data pair, and k is an effect coefficient determined based on a ratio between the rescue effect when the out-of-hospital cardiac arrest patient is in the post-cardiac arrest unresponsive state and the rescue effect when the out-of-hospital cardiac arrest patient is in the cardiac arrest onset state.

In an embodiment of the disclosure, an apparatus for assessing work performance of a target organization is provided, and the apparatus includes a first acquisition module, a second acquisition module, and an assessment module.

The first acquisition module is configured to acquire at least one set of witness information data pairs within a business coverage area of the target organization and within a preset historical time period; each set of the witness information data pairs includes a witness rate and a witness action rate, and corresponds to a disease process state of a target patient; the witness rate is a percentage of target patients in a certain disease process state being witnessed; and the witness action rate is a proportion of witnesses, among all witnesses, who take preset rescue measures upon discovering the target patient in a certain disease process state, and the witnesses belong to a target population.

The second acquisition module is configured to calculate a rescue action conversion efficiency corresponding to the target organization based on the at least one set of witness information data pairs; the rescue action conversion efficiency is a proportion, among all target patients in any disease process state, of target patients who receive effective preset rescue measures from members of the target population.

The assessment module is configured to assess the work performance of the target organization based on the rescue action conversion efficiency corresponding to the target organization.

In some embodiments, the first acquisition module is embodied by software stored in at least one memory and executable by at least one processor; likewise, the second acquisition module is embodied by software stored in at least one memory and executable by at least one processor; likewise, the assessment module is embodied by software stored in at least one memory and executable by at least one processor.

In an embodiment of the disclosure, an electronic device is provided, the electronic device includes a memory and a processor, the memory stores a computer program, and the processor, when running the program, executes the above method.

In an embodiment of the disclosure, a storage medium is provided, the storage medium is configured to store a computer-readable program, when the computer-readable program is run, the storage medium executes the above method.

The technical solutions provided in the embodiments of the disclosure have at least the following advantages compared with the prior art.

In the embodiments provided by the disclosure, at least one set of witness information data pairs composed of a witness rate and a witness action rate is acquired within a business coverage area of the target organization and within a preset historical time period; a rescue action conversion efficiency corresponding to the target organization is calculated based on the at least one set of witness information data pairs; and the work performance of the target organization is evaluated based on the rescue action conversion efficiency corresponding to the target organization. Since the rescue action conversion efficiency can accurately represent the proportion of target patients receiving effective preset rescue measures from members of the target population among all target patients in any given disease process state, and this proportion is related to the work performance of medical rescue-related organizations, the embodiments provided by the disclosure can achieve effective evaluation of medical rescue-related organizations.

### BRIEF DESCRIPTION OF DRAWINGS

The disclosure will be further illustrated by exemplary embodiments, which will be described in detail through the accompanying drawings. These embodiments are not limiting, and in these embodiments, the same numerals represent the same structures.

FIG. 1 illustrates a flowchart of a method for assessing work performance of a target organization according to some embodiments of the disclosure.

FIG. 2 illustrates a schematic diagram showing roles and transitions of bystanders before emergency medical services arrive at the scene according to some embodiments of the disclosure.

FIG. 3 illustrates a schematic diagram of an apparatus for assessing work performance of a target organization according to some embodiments of the disclosure.

FIG. 4 illustrates a flowchart of another method for assessing work performance of a target organization according to some embodiments of the disclosure.

FIG. 5 illustrates a schematic structural diagram of an electronic device according to some embodiments of the disclosure.

### DETAILED DESCRIPTION OF EMBODIMENTS

To illustrate the technical solutions of the embodiments of the disclosure more clearly, the following briefly introduces the accompanying drawings required for describing the embodiments. Apparently, the accompanying drawings in the following description show only some examples or embodiments of the disclosure. To those of ordinary skill in the art, other similar scenarios may be applied to the disclosure based on these drawings without creative efforts. Unless obvious from the context or otherwise stated, the same reference numerals in the drawings represent the same structures or operations.

It should be understood that the terms “system”, “apparatus”, “unit”, and/or “module” used herein are methods used to distinguish different components, elements, parts, sections, or assemblies at different levels. However, if other words can achieve the same purpose, they may be replaced by other expressions.

As used in this disclosure and the claims, unless the context clearly indicates an exception, the terms “a”, “an”, “one”, and/or “the” are not intended to refer solely to the singular but may also include the plural. In general, the terms “comprise” and “include” only indicate the inclusion of explicitly identified steps and elements. These steps and elements do not form an exhaustive enumeration, and the method or apparatus may also contain other steps or elements.

Flowcharts are used in the disclosure to illustrate the operations performed by the system according to the embodiments of the disclosure. It should be understood that preceding or following operations do not necessarily need to be executed precisely in sequence. On the contrary, the various steps may be processed in reverse order or simultaneously. Furthermore, other operations may be added to these processes, or one or several operations may be removed from these processes.

To facilitate understanding, the technical solutions of the disclosure are described below in conjunction with the accompanying drawings and embodiments.

FIG. 1 illustrates a flowchart of a method for assessing work performance of a target organization according to some embodiments of the disclosure. As shown in FIG. 1, the method for assessing the work performance of the target organization includes the following steps S110 to S130.

In step S110, at least one set of witness information data pairs within a business coverage area of the target organization and within a preset historical time period is acquired, each set of witness information data pairs includes a witness rate and a witness action rate, and each set of witness information data pairs corresponds to a disease process state of target patients.

The target organization can be an organization related to emergency medical rescue tasks. In some embodiments, the target organization is an emergency medical rescue training organization. The emergency medical rescue training organization recruits a large number of trainees for training. The purpose of the training is to enable more people who may witness cardiac arrest patients to have the ability to participate in emergency medical rescue operations. An accurately quantified assessment result of work performance can provide references for the emergency medical rescue training organizations to carry out their business, which is conducive to promoting the long-term stable development of such organizations, thereby improving the public's first aid level. In contrast, the prior art cannot quantitatively assess the work performance of emergency medical rescue training organizations.

In some embodiments, one set of witness information data pairs corresponds to one disease process state of the target patient, and includes one witness rate and one witness action rate.

Each target patient can be a patient requiring emergency medical rescue. For example, the target patient can be an out-of-hospital cardiac arrest patient. As another example, the target patient can be a stroke patient.

The disease process state is a certain state of the patient during the onset of the disease. For example, for an out-of-hospital cardiac arrest patient, the disease process state may include: cardiac arrest onset state, post-cardiac arrest unresponsive state, etc.

The witness rate can be defined as: the percentage of target patients in a certain disease process state being witnessed.

In some embodiments, the witnesses belong to a target population. The target population can be trainees of an emergency rescue training organization, employees of large enterprises and organizations, residents of a certain administrative area, or other groups, and is not limited by the expressions in this specification.

In some embodiments, the witness rate can be obtained based on the number of target patients witnessed while in a certain disease process state and the total number of target patients in that certain disease process state.

As an example only, the target patients are out-of-hospital cardiac arrest patients and the disease process state is the cardiac arrest onset state, the witness rate can be obtained by: obtaining a first witness rate based on the ratio between the number of out-of-hospital cardiac arrest patients witnessed while in the cardiac arrest onset state and the total number of out-of-hospital cardiac arrest patients in the cardiac arrest onset state, and the first witness rate is expressed by the following formula:

$$First Witness Rate = Number of OHCA patients witnessed while in cardiac arrest onset state / Total number of OHCA patients in cardiac arrest onset state \qquad ( 1 )$$

In the case where the disease process state is the post-cardiac arrest unresponsive state, the witness rate can be obtained by: obtaining a second witness rate based on the ratio between the number of out-of-hospital cardiac arrest patients witnessed while already in the post-cardiac arrest unresponsive state and the total number of out-of-hospital cardiac arrest patients in the post-cardiac arrest unresponsive state, and the second witness rate is expressed by the following formula:

$$Second Witness Rate = Number of OHCA patients witnessed while already in post - cardiac arrest unresponsive state / Total number of OHCA patients in post - cardiac arrest unresponsive state \qquad ( 2 )$$

The witness action rate can be defined as: the proportion of witnesses, among all witnesses, who take preset rescue measures upon discovering the target patient in a certain disease process state, and the witnesses belong to a target population.

In some embodiments, the witness action rate can be obtained based on the number of witnesses who take preset rescue measures when the target patient is in a certain disease process state and the total number of witnesses.

As an example only, the disease process state is the cardiac arrest onset state, the witness action rate can be obtained by: obtaining a first witness action rate based on the ratio between the number of witnesses who take preset rescue measures when the target patient is in the cardiac arrest onset state and the total number of witnesses who witness the target patient in the cardiac arrest onset state, and the first witness action rate is expressed by the following formula:

$$First Witness Action Rate = Number of witnesses who take preset rescue measures / Total number of witnesses who witness the target patient in cardiac arrest onset state \qquad ( 3 )$$

The preset rescue measures include one or more combinations of: manual cardiopulmonary resuscitation and use of an automated external defibrillator.

In specific implementation, the aforementioned preset rescue measures can be further subdivided to obtain more targeted rescue action conversion efficiency. For example, referring to relevant CPR guidelines, manual CPR operations can be further subdivided according to the depth and frequency of chest compressions achieved, resulting in multiple types of manual CPR operations classified by quality of first aid action. Consequently, the rescue action conversion efficiency corresponding to these multiple types of manual CPR operations classified by quality can be obtained.

The above embodiments are examples only. In cases where the target patient is another type of patient, for example, a stroke patient, the preset rescue measures are other corresponding rescue measures, and are not limited by the expressions in this specification.

In the case where the disease process state is the post-cardiac arrest unresponsive state, the witness action rate can be obtained by: obtaining a second witness action rate included in the witness information data pair based on the ratio between the number of witnesses who take preset rescue measures when the target patient is in the post-cardiac arrest unresponsive state and the total number of witnesses who witness the out-of-hospital cardiac arrest patient in the post-cardiac arrest unresponsive state, and the second witness action rate is expressed by the following formula:

$$Second Witness Action Rate = Number of witnesses who take preset rescue measures / Total number of witnesses who witness the OHCA patient in post - cardiac arrest unresponsive state \qquad ( 4 )$$

In step S120, a rescue action conversion efficiency corresponding to the target organization is calculated based on the at least one set of witness information data pairs.

The rescue action conversion efficiency is the proportion, among all target patients in any disease process state, of target patients who receive effective preset rescue measures from members of the target population.

In some embodiments, the rescue action conversion efficiency corresponding to the target organization can be calculated by: for each set of witness information data pairs, calculating the product of the witness rate and the witness action rate in that set of witness information data pairs; and obtaining the rescue action conversion efficiency based on the products of the witness rates and the witness action rates from all sets of witness information data pairs, and the rescue action conversion efficiency is expressed by the following formula:

$$T = {\sum }_{i = 1}^{n} {k}_{i} {w}_{i} * {t}_{i} \qquad ( 5 )$$

where T is the rescue action conversion efficiency, wᵢ is the witness rate in the i-th set of witness information data pairs, tᵢ is the witness action rate in the i-th set of witness information data pairs, n is the number of the at least one set of witness information data pairs, and kᵢ is an effect coefficient corresponding to the i-th set of witness information data pairs, and the effect coefficient is used to represent the effect of being administered the preset rescue measures when the target patient is in a certain disease process state.

In some embodiments, the target patient is an out-of-hospital cardiac arrest patient; the at least one set of witness information data pairs includes: a first witness information data pair and a second witness information data pair; the first witness information data pair corresponds to the cardiac arrest onset state of the out-of-hospital cardiac arrest patient; the second witness information data pair corresponds to the post-cardiac arrest unresponsive state of the out-of-hospital cardiac arrest patient; the calculation formula for the rescue action conversion efficiency corresponding to the target organization can be as follows:

$$T = {w}_{1} * {t}_{1} + {kw}_{2} * {t}_{2} \qquad ( 6 )$$

where T is the rescue action conversion efficiency corresponding to the target organization, w₁ is the witness rate in the first witness information data pair, t₁ is the witness action rate in the first witness information data pair, w₂ is the witness rate in the second witness information data pair, t₂ is the witness action rate in the second witness information data pair, and k is an effect coefficient determined based on a ratio between the rescue effect when the out-of-hospital cardiac arrest patient is in the post-cardiac arrest unresponsive state and the rescue effect when the out-of-hospital cardiac arrest patient is in the cardiac arrest onset state. In specific implementation, k can be replaced by the ratio of survival rates (such as return of spontaneous circulation, survival to discharge, or survival to 30 days), and its value range is (0, 1).

According to formula (6), the rescue action conversion efficiency corresponding to the target organization can be improved by increasing the number of witnesses when the out-of-hospital cardiac arrest patient is in the onset state (e.g., by reducing the rate of living alone) and/or by increasing the number of people with the skills to take preset rescue measures (by increasing the number of trainees in emergency medical rescue training organizations).

Generally speaking, the rescue effect is best when the out-of-hospital cardiac arrest patient is in the cardiac arrest onset state, and the rescue effect is poorer when the patient is in the post-cardiac arrest unresponsive state. Therefore, the effect coefficient is used to reflect the rescue efficiency when the out-of-hospital cardiac arrest patient is in different disease process states. In the embodiments provided by the disclosure, the work performance of the target organization can be assessed based on the rescue action conversion efficiency. To improve the rescue action conversion efficiency, according to formula (6), a more effective way is to improve the rescue action conversion efficiency when the out-of-hospital cardiac arrest patient is in the cardiac arrest onset state. For example, the recruitment and training of trainees in densely populated areas can be strengthened, so that out-of-hospital cardiac arrest patients in the cardiac arrest onset state can be discovered in a timely manner by persons capable of implementing rescue measures.

For example, a 120 emergency network serving a population of 500,000 in a certain area registers 800 cases of out-of-hospital cardiac arrest annually. Among these, the total number of OHCA patients in the cardiac arrest onset state is 400 (this data is obtained statistically by the emergency medical rescue organization after being alerted by a third party who discovered the patient in cardiac arrest onset state and after their personnel arrive on scene). The number of patients witnessed while in cardiac arrest onset state is 160 cases, among which 16 witnesses performed CPR at the scene. Then, according to formula (1), the first witness rate w₁ is 40%, and according to formula (3), the first witness action rate t₁ is 10%. The total number of OHCA patients in the post-cardiac arrest unresponsive state is 400 cases. The number of cases witnessed while in the post-cardiac arrest unresponsive state is 200 cases, among which 10 witnesses performed CPR at the scene. Then, according to formula (2), the second witness rate w₂ is 50%, and according to formula (4), the second witness action rate t₂ is 5%. Assuming k=0.1, then according to formula (6), the rescue action conversion efficiency T corresponding to the target organization is 4.25%.

Based on the above calculation, the witness rate for OHCA patients in the disease process state in the area covered by this 120 system is at a currently acceptable average level, but the witness action rate is low, indicating that only a very low proportion of witnesses of OHCA patients in the disease process state in this area participate in effective on-site first aid. The main reason for this phenomenon may be that the target organization in this area has not provided adequate training to its trainees, resulting in insufficient conversion from witnesses to actors. Therefore, the next key task for this target organization should be to strengthen the quality of training.

The development level of public first aid for out-of-hospital cardiac arrest varies greatly among countries and areas. In specific implementation, the calculation formula for the rescue action conversion efficiency corresponding to the target organization provided in the embodiments of the disclosure can be applied in stages according to the development level from low to high. For example, when the rescue action conversion efficiency for performing CPR on OHCA patients is low (e.g., below 5%), the focus should be on improving the conversion efficiency for CPR implementation. After a considerable proportion of the OHCA population can receive public CPR, further strengthening can be continued by either improving the rescue action conversion efficiency for AED application on OHCA patients or enhancing the quality of CPR performed on OHCA patients.

In step S130, the work performance of the target organization is assessed based on the rescue action conversion efficiency corresponding to the target organization.

In specific implementation, the work performance of the target organization can be assessed based on the rescue action conversion efficiency corresponding to the target organization in various ways.

In some embodiments, multiple score thresholds can be preset. The rescue action conversion efficiency corresponding to the target organization is compared with these preset score thresholds, and the work performance assessment of the target organization is determined based on the comparison result. For example, if the rescue action conversion efficiency corresponding to the target organization is greater than a preset pass score threshold, the work performance of the target organization can be assessed as pass; if it is greater than a preset good score threshold, it can be assessed as good; if it is greater than a preset excellent score threshold, it can be assessed as excellent.

In some embodiments, the work performance of the target organization can be assessed by comparing its rescue action conversion efficiency with that of other organizations. For example, the rescue action conversion efficiencies corresponding to multiple organizations, including the target organization, can be sorted, and the work performance of the target organization can be determined based on the sorting result.

In some embodiments, a frequency table of rescue action conversion efficiencies can be constructed based on multiple rescue action conversion efficiencies including the one corresponding to the target organization; and each rescue action conversion efficiency corresponds to one organization.

In specific implementation, the aforementioned rescue action conversion efficiencies can be arranged in increasing order to obtain the frequency table. The percentile corresponding to the target organization can be calculated based on this frequency table using the following formula:

$${P}_{x} = L + \frac{i}{{F}_{L + i} - {F}_{L}} ( n \cdot x \% ) - {F}_{L} \qquad ( 7 )$$

where Pₓ is the percentile corresponding to the x-th rescue action conversion efficiency in the frequency table, L is the lower limit of the class interval where Pₓ is located in the frequency table, i is the class width of that interval, F_{L} is the cumulative frequency up to L, F_{L+i} is the cumulative frequency up to L+i, F_{L+i}−F_{L} is the frequency within that class interval, and n is the total frequency.

As an example only, assume there are 20 similar 120 emergency networks in different areas, whose corresponding rescue action conversion efficiencies (%) are: 4.25, 3.78, 6.12, 7.89, 2.21, 1.34, 0.85, 14.52, 9.97, 3.22, 4.55, 2.12, 1.10, 5.46, 5.98, 3.00, 0.21, 1.32, 4.09, 8.16. According to formula (7), P25, P50, and P75 can be calculated as 1.73, 3.94, and 6.05 respectively (for convenience, the data can be obtained using statistical packages like SAS).

After calculating the percentile corresponding to the target organization, a work performance score for the target organization can be obtained based on the percentile, and then the work performance of the target organization can be assessed through this score. For example, the work performance can be assessed by defining cut points based on percentiles. All participating organizations can be divided into four tiers using P₂₅, P₅₀, and P₇₅ as cut points. Comparing the work performance among multiple organizations can incentivize underperformers to actively learn from top performers, thereby comprehensively improving work performance in multiple aspects.

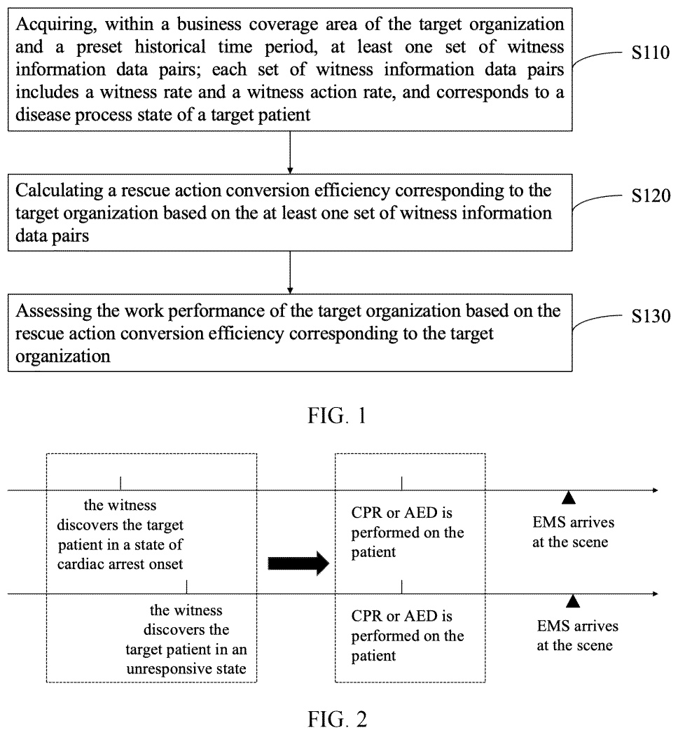

FIG. 2 shows the process of emergency rescue measures being taken for an out-of-hospital cardiac arrest patient before emergency medical services arrive at the scene. As can be seen from FIG. 2, for target patients requiring emergency medical measures, whether they survive when in a disease process state depends on two core elements: first, whether there is a witness when the target patient is in the disease process state, and second, whether the witness at the scene can immediately take corresponding emergency rescue measures when the target patient is in the disease process state. In the embodiments provided by the disclosure, the witness rate reflects the percentage of target patients in a certain disease process state being witnessed; the witness action rate reflects the proportion, among all witnesses, of witnesses who take effective preset rescue measures upon discovering the target patient in a certain disease process state; the rescue action conversion efficiency corresponding to the target organization is obtained based on the witness rate and the witness action rate; and the work performance of the target organization is quantitatively assessed based on this rescue action conversion efficiency. This can promote the target organization to improve its work, enabling more witnesses to take emergency rescue measures for target patients in a disease process state, thereby giving the target patients a greater chance of survival.

FIG. 3 illustrates a schematic diagram of an apparatus for assessing work performance of a target organization according to some embodiments of the disclosure.

As shown in FIG. 3, the apparatus for assessing the work performance of the target organization includes: a first acquisition module 310, a second acquisition module 320, and an assessment module 330.

The first acquisition module 310 is configured to acquire, within a business coverage area of the target organization and a preset historical time period, at least one set of witness information data pairs; each set of the witness information data pairs includes a witness rate and a witness action rate, and corresponds to a disease process state of a target patient; the witness rate is a percentage of target patients in a certain disease process state being witnessed; the witness action rate is a proportion of witnesses, among all witnesses, who take preset rescue measures upon discovering the target patient in a certain disease process state; and the witnesses belong to a target population.

The second acquisition module 320 is configured to calculate a rescue action conversion efficiency corresponding to the target organization based on the at least one set of witness information data pairs; and the rescue action conversion efficiency is a proportion, among all target patients in any disease process state, of target patients who receive effective preset rescue measures from members of the target population.

The assessment module 330 is configured to assess the work performance of the target organization based on the rescue action conversion efficiency corresponding to the target organization.

In some embodiments, the witness rate is obtained by: obtaining the witness rate based on the number of target patients witnessed while in a certain disease process state and the total number of target patients in that certain disease process state.

In some embodiments, the target patients are out-of-hospital cardiac arrest patients; the disease process state is a cardiac arrest onset state, and the obtaining the witness rate based on the number of target patients witnessed while in a certain disease process state and the total number of target patients in that certain disease process state includes: obtaining a first witness rate based on a ratio between the number of out-of-hospital cardiac arrest patients witnessed while in the cardiac arrest onset state and the total number of out-of-hospital cardiac arrest patients in the cardiac arrest onset state; or the disease process state is a post-cardiac arrest unresponsive state, and the obtaining the witness rate based on the number of target patients witnessed while in a certain disease process state and the total number of target patients in that certain disease process state includes: obtaining a second witness rate based on a ratio between the number of out-of-hospital cardiac arrest patients witnessed while already in the post-cardiac arrest unresponsive state and the total number of out-of-hospital cardiac arrest patients in the post-cardiac arrest unresponsive state.

In some embodiments, the witness action rate is obtained by: obtaining the witness action rate based on the number of witnesses who take preset rescue measures when the target patient is in a certain disease process state and the total number of witnesses.

In some embodiments, the target patient is an out-of-hospital cardiac arrest patient; the disease process state is a cardiac arrest onset state, and the obtaining the witness action rate based on the number of witnesses who take preset rescue measures when the target patient is in a certain disease process state and the total number of witnesses includes: obtaining a first witness action rate based on a ratio between the number of witnesses who take preset rescue measures when the out-of-hospital cardiac arrest patient is in the cardiac arrest onset state and the total number of witnesses who witness the out-of-hospital cardiac arrest patient in the cardiac arrest onset state; or the disease process state is a post-cardiac arrest unresponsive state, and the obtaining the witness action rate based on the number of witnesses who take preset rescue measures when the target patient is in a certain disease process state and the total number of witnesses includes: obtaining a second witness action rate based on a ratio between the number of witnesses who take preset rescue measures when the out-of-hospital cardiac arrest patient is in the post-cardiac arrest unresponsive state and the total number of witnesses who witness the out-of-hospital cardiac arrest patient in the post-cardiac arrest unresponsive state; and the preset rescue measures include one or more combinations of: manual cardiopulmonary resuscitation and use of an AED.

In some embodiments, the calculating the rescue action conversion efficiency corresponding to the target organization based on the at least one set of witness information data pairs includes: for each set of the witness information data pairs, calculating a product of the witness rate and the witness action rate in that set of witness information data pairs; and obtaining the rescue action conversion efficiency based on the products of the witness rates and the witness action rates from all sets of witness information data pairs.

In some embodiments, the rescue action conversion efficiency is obtained by the following calculation formula:

$$T = {\sum }_{i = 1}^{n} {k}_{i} {w}_{i} * {t}_{i}$$

where T is the rescue action conversion efficiency, wᵢ is the witness rate in the i-th set of witness information data pairs, tᵢ is the witness action rate in the i-th set of witness information data pairs, n is the number of the at least one set of witness information data pairs, and kᵢ is an effect coefficient corresponding to the i-th set of witness information data pairs, the effect coefficient is used to represent the effect of being administered the preset rescue measures when the target patient is in a certain disease process state.

In some embodiments, the target patient is an out-of-hospital cardiac arrest patient; the at least one set of witness information data pairs includes: a first witness information data pair and a second witness information data pair; the first witness information data pair corresponds to the cardiac arrest onset state of the out-of-hospital cardiac arrest patient; the second witness information data pair corresponds to the post-cardiac arrest unresponsive state of the out-of-hospital cardiac arrest patient; the calculation formula for the rescue action conversion efficiency corresponding to the target organization is as follows:

$$T = {w}_{1} * {t}_{1} + {kw}_{2} * {t}_{2}$$

where T is the rescue action conversion efficiency corresponding to the target organization, w₁ is the witness rate in the first witness information data pair, t₁ is the witness action rate in the first witness information data pair, w₂ is the witness rate in the second witness information data pair, t₂ is the witness action rate in the second witness information data pair, and k is an effect coefficient determined based on a ratio between the rescue effect when the out-of-hospital cardiac arrest patient is in the post-cardiac arrest unresponsive state and the rescue effect when the out-of-hospital cardiac arrest patient is in the cardiac arrest onset state, and the effect coefficient is used to represent the effect of being administered the preset rescue measures when the target patient is in a certain disease process state.

In some embodiments, the assessing the work performance of the target organization based on the rescue action conversion efficiency corresponding to the target organization includes: constructing a rescue action conversion efficiency frequency table based on multiple rescue action conversion efficiencies including the rescue action conversion efficiency corresponding to the target organization; each rescue action conversion efficiency in the multiple rescue action conversion efficiencies corresponds to one organization; obtaining a percentile corresponding to the target organization based on the frequency table of rescue action conversion efficiencies; and obtaining a work performance score for the target organization based on the percentile.

In the embodiments of the aforementioned apparatus for assessing the work performance of the target organization, the specific processing of each module and the technical effects brought thereby can be referred to the relevant descriptions in the corresponding method embodiments, and are not repeated here.

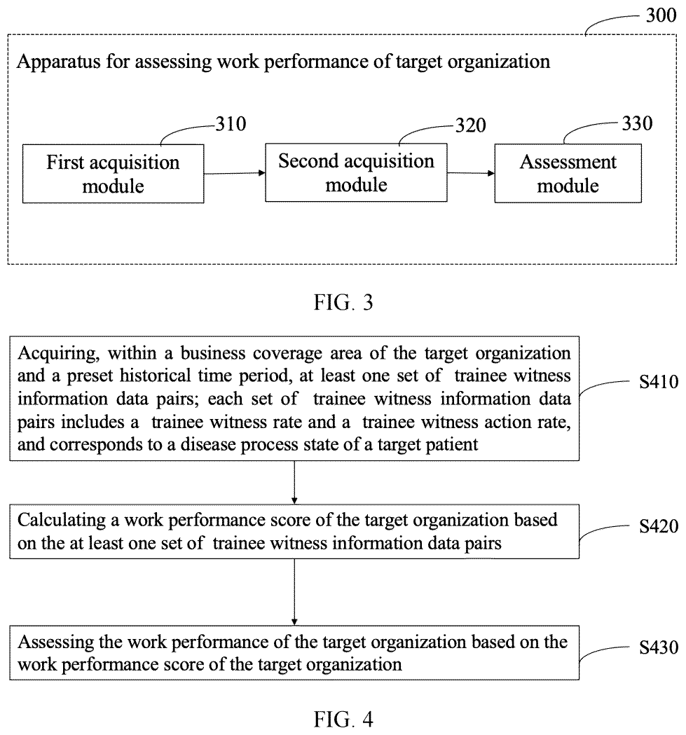

FIG. 4 illustrates a flowchart of another method for assessing work performance of a target organization according to some embodiments of the disclosure. As shown in FIG. 4, the method includes the following steps.

In step S410, at least one set of trainee witness information data pairs is acquired within a business coverage area of the target organization and within a preset historical time period. Each set of trainee witness information data pairs includes a trainee witness rate and a trainee action rate.

In some embodiments, each set of trainee witness information data pairs corresponds to a disease process state of a target patient, and includes one trainee witness rate and one trainee action rate.

For detailed descriptions of the target organization, target patient, and disease process state, refer to the relevant content in FIG. 1, which will not be repeated here.

To obtain valid statistical data, the trainee witness information data pairs within the business coverage area of the target organization and within the preset historical time period can be acquired. For example, if the target organizations are n numbers of emergency medical rescue training organizations C₁, C₂, . . . , Cₙ located in city A, multiple sets of trainee witness information data pairs for the previous year can be acquired for each emergency medical rescue training organization Cᵢ (where i is a value in the range 1 to n) in the city A. Using these multiple sets of trainee witness information data pairs, the work performance of the emergency medical rescue training organization Cᵢ in the previous year can be assessed.

In some embodiments, the trainee witness rate can be: the proportion, among all trainees trained by the target organization, of trainees who witness the target patient in the certain disease process state.

As an example only, the target patient is an out-of-hospital cardiac arrest patient.

In some embodiments, the disease process state is the cardiac arrest onset state, the trainee witness rate can be obtained by: obtaining a first trainee witness rate based on the ratio between the number of trainees, among those trained by the target organization, who witness the target patient in the cardiac arrest onset state and the total number of trainees trained by the target organization. The first trainee witness rate is expressed by the following formula:

$$First Trainee Witness Rate = Number of trainees who witness target patient in cardiac arrest onset state / Total number of trainees trained by the target organization \qquad ( 8 )$$

In some embodiments, the disease process state is the post-cardiac arrest unresponsive state, the trainee witness rate can be obtained by: obtaining a second trainee witness rate based on the ratio between the number of trainees, among those trained by the target organization, who witness the target patient in the post-cardiac arrest unresponsive state and the total number of trainees trained by the target organization. The second trainee witness rate is expressed by the following formula:

$$Second Trainee Witness Rate = Number of trainees who witness target patient in post - cardiac arrest unresponsive state / Total number of trainees trained by the target organization \qquad ( 9 )$$

In some embodiments, the trainee witness action rate can be: the proportion, among all trainees trained by the target organization, of trainees trained by the target organization who take preset rescue measures upon discovering the target patient in the certain disease process state.

As an example only, the target patient is an out-of-hospital cardiac arrest patient.

In some embodiments, the disease process state is the cardiac arrest onset state, the trainee action rate can be obtained by: obtaining a first trainee action rate based on the ratio between the number of trainees trained by the target organization who take preset rescue measures upon discovering the target patient in the cardiac arrest onset state and the total number of all trainees trained by the target organization. The first trainee action rate is expressed by the following formula:

$$First Trainee Action Rate = Number of trainees who take preset rescue measures for target patient in cardiac arrest onset state / Total number of all trainees trained by the target organization \qquad ( 10 )$$

In some embodiments, the disease process state is the post-cardiac arrest unresponsive state, the trainee action rate is obtained by: obtaining a second trainee action rate based on the ratio between the number of trainees trained by the target organization who take preset rescue measures upon discovering the target patient in the post-cardiac arrest unresponsive state and the total number of all trainees trained by the target organization. The second trainee action rate is expressed by the following formula:

$$Second Trainee Action Rate = Number of trainees who take preset rescue measures for target patient in post - cardiac arrest unresponsive state / Total number of all trainees trained by the target organization \qquad ( 11 )$$

In specific implementation, the preset rescue measures include one or more combinations of: manual cardiopulmonary resuscitation and use of an automated external defibrillator. For a detailed description of the preset rescue measures, refer to the relevant content in FIG. 1, which will not be repeated here.

In step S420, a work performance score for the target organization is calculated based on the at least one set of trainee witness information data pairs.

In specific implementation, for each set of trainee witness information data pairs, the product of the trainee witness rate and the trainee action rate in that set can be calculated; and the work performance score for the target organization is obtained based on the products of the trainee witness rates and the trainee action rates from all sets of trainee witness information data pairs.

In some embodiments, the target patient is an out-of-hospital cardiac arrest patient. The at least one set of trainee witness information data pairs includes: a first trainee witness information data pair and a second trainee witness information data pair; the first trainee witness information data pair corresponds to the cardiac arrest onset state; and the second trainee witness information data pair corresponds to the post-cardiac arrest unresponsive state. The calculation formula for the work performance score of the target organization is as follows:

$$F = {a}_{1} * {b}_{1} + {ka}_{2} * {b}_{2} \qquad ( 12 )$$

where F is the work performance score of the target organization, a₁ is the trainee witness rate in the first trainee witness information data pair, b₁ is the trainee action rate in the first trainee witness information data pair, a₂ is the trainee witness rate in the second trainee witness information data pair, b₂ is the trainee action rate in the second trainee witness information data pair, and k is an effect coefficient determined based on a ratio between the rescue effect when the out-of-hospital cardiac arrest patient is in the post-cardiac arrest unresponsive state and the rescue effect when the out-of-hospital cardiac arrest patient is in the cardiac arrest onset state.

For a detailed description of the effect coefficient, refer to the relevant content in FIG. 1, which will not be repeated here.

In specific implementation, the effect coefficient corresponding to the target patient being in the post-cardiac arrest unresponsive state is low (because the rescue effect is poorer at this time). According to formula (12), to improve the work performance score of the target organization, a more effective way is to increase the trainee witness rate when the target patient is in the cardiac arrest onset state. For example, the target organization can increase the trainee witness rate by expanding the recruitment of trainees in densely populated areas.

As an example only, a certain CPR training organization trains 1000 trainees per year. Among them, the number of trainee encounters where a trainee witnessed an OHCA patient in cardiac arrest onset state is 10, and 4 encounters involved the trainee performing CPR on the patient. Then, according to formula (8), the first trainee witness rate is 1%, and according to formula (10), the first trainee action rate is 40%. Furthermore, the number of trainee encounters where a trainee witnessed an OHCA patient in post-cardiac arrest unresponsive state is 20, and 10 encounters involved the trainee performing CPR on the patient. Then, according to formula (9), the second trainee witness rate is 2%, and according to formula (11), the second trainee action rate is 50%. Assuming k=0.1, according to formula (12), the work performance score of the target organization is 0.5%.

From the above example, it can be seen that a high proportion of the trainees trained by this organization participate in effective on-site rescue when encountering an out-of-hospital cardiac arrest patient. However, the prominent problem is that the trainees of this organization rarely encounter patients. This indicates that the organization has deficiencies in the recruitment and screening of target trainees, failing to identify potential bystanders, thus resulting in a relatively high amount of ineffective training. Therefore, the next key task should be the recruitment and screening of trainees.

In step S430, the work performance of the target organization is assessed based on the work performance score of the target organization.

In specific implementation, the work performance of the target organization can be assessed based on its work performance score in various ways.

In some embodiments, multiple score thresholds can be preset. The work performance score of the target organization is compared with these preset score thresholds, and the work performance assessment of the target organization is determined based on the comparison result.

In some embodiments, the work performance of the target organization can be assessed by comparing its work performance score with those of other organizations. For example, the work performance scores of multiple organizations, including the target organization, can be sorted, and the work performance of the target organization can be determined based on the sorting result.

In some embodiments, a frequency table of work performance scores can be constructed based on multiple work performance scores including that of the target organization. The work performance of the target organization is assessed based on this frequency table; and each work performance score corresponds to one organization.

For a detailed description of assessing the work performance of the target organization based on the frequency table of work performance scores, refer to the relevant content in FIG. 1 regarding assessing the work performance of the target organization based on the frequency table of rescue action conversion efficiencies, which will not be repeated here.

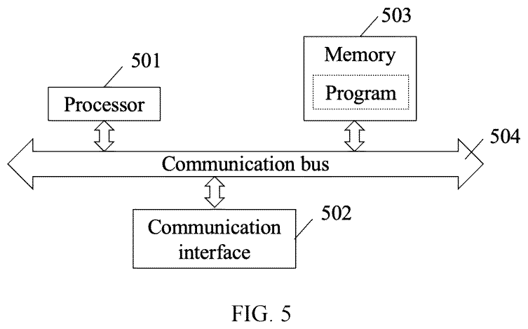

FIG. 5 illustrates a schematic structural diagram of an electronic device according to some embodiments of the disclosure.

As shown in FIG. 5, the electronic device includes: at least one processor 501, at least one communication interface 502, at least one memory 503, and at least one communication bus 504; optionally, the communication interface 502 can be an interface of a communication module, such as a global system for mobile communications module (GSM) interface; the processor 501 may be a central processing unit (CPU), or an application specific integrated circuit (ASIC), or one or more integrated circuits configured to implement the embodiments of the disclosure. The memory 503 may include high-speed random access memory (RAM), and may also include non-volatile memory, such as at least one disk storage. The memory 503 stores a program, and the processor 501 invokes the program stored in the memory 503 to execute some or all of the method embodiments described above.

The disclosure relates to a storage medium, used for storing a computer-readable program, when the computer-readable program is executed, some or all of the method embodiments described above are performed.

Optionally, the storage medium may be a non-transitory computer-readable storage medium, for example, the non-transitory computer-readable storage medium may be read-only memory (ROM), RAM, compact disc read-only memory (CD-ROM), magnetic tape, floppy disk, optical data storage device, etc.

Based on the same inventive concept, an embodiment of the disclosure also provides a computer program product, including a computer program, when the program is executed by a processor, it implements some or all of the method embodiments described above.

The basic concepts have been described above. Apparently, to a person skilled in the art, the above detailed disclosure is merely illustrative and does not constitute a limitation on the disclosure. Although not explicitly stated herein, those skilled in the art may make various modifications, improvements, and amendments to the disclosure. Such modifications, improvements, and amendments are suggested in the disclosure, and therefore still fall within the spirit and scope of the exemplary embodiments of the disclosure.

Furthermore, the disclosure uses specific words to describe its embodiments. Terms such as “one embodiment”, “an embodiment”, and/or “some embodiments” mean that a certain feature, structure, or characteristic related to at least one embodiment of the disclosure. Therefore, it should be emphasized and noted that “an embodiment” or “one embodiment” or “an alternative embodiment” mentioned twice or more in different places in the disclosure does not necessarily refer to the same embodiment. In addition, some features, structures, or characteristics in one or more embodiments of the disclosure may be appropriately combined.

Moreover, unless explicitly stated in the claims, the order of processing elements and sequences, the use of numbers and letters, or the use of other names in the disclosure is not intended to limit the order of the processes and methods of the disclosure. Although the foregoing disclosure has discussed some embodiments currently considered useful through various examples, it should be understood that these details are for illustrative purposes only, and the appended claims are not limited to the disclosed embodiments. On the contrary, the claims are intended to cover all modifications and equivalent combinations that conform to the essence and scope of the embodiments of the disclosure. For example, although the system components described above can be implemented by hardware devices, they can also be implemented solely through software solutions, such as installing the described system on existing servers or mobile devices.

Similarly, it should be noted that, in order to simplify the expression of the disclosure and thereby facilitate the understanding of one or more inventive embodiments, the description of the embodiments of the disclosure sometimes groups multiple features into one embodiment, drawing, or its description. However, this method of disclosure does not mean that the object of the disclosure requires more features than those mentioned in the claims. In fact, the features of the embodiments are fewer than all the features of the single embodiments disclosed above.

Some embodiments use numbers describing quantities of components or attributes. It should be understood that such numbers used in the description of the embodiments are, in some instances, modified by the terms “about”, “approximately”, or “substantially”. Unless otherwise stated, “about”, “approximately”, or “substantially” indicates that the number allows a variation of +20%. Accordingly, in some embodiments, the numerical parameters used in the specification and claims are all approximate values, which may vary according to the characteristics required by individual embodiments. In some embodiments, the numerical parameters should take into account the specified significant digits and apply the method of general digit retention. Although the numerical ranges and parameters used to confirm their breadth in some embodiments of the disclosure are approximate values, in specific embodiments, the setting of such numerical values is as precise as possible within the feasible range.

For each patent, patent application, patent application publication, and other materials, such as articles, books, specifications, publications, documents, etc., cited in the disclosure, their entire contents are hereby incorporated into the disclosure by reference. Excluded are application history documents that are inconsistent with or conflict with the content of the disclosure, and documents that limit the broadest scope of the claims of the disclosure (whether currently attached or attached later to the disclosure. It should be noted that if there is any inconsistency or conflict between the descriptions, definitions, and/or terminology used in the ancillary materials of the disclosure and the content described in the disclosure, the descriptions, definitions, and/or terminology used in the disclosure shall prevail.

Finally, it should be understood that the embodiments described in the disclosure are only used to illustrate the principles of the embodiments of the disclosure. Other variations may also fall within the scope of the disclosure. Therefore, by way of example and not limitation, alternative configurations of the embodiments of the disclosure can be considered consistent with the teachings of the disclosure. Accordingly, the embodiments of the disclosure are not limited to the embodiments explicitly introduced and described herein.

1. A method for assessing work performance of a target organization, comprising:
acquiring, within a business coverage area of the target organization and a preset historical time period, at least one set of witness information data pairs; wherein each set of witness information data pairs comprises a witness rate and a witness action rate, each set of witness information data pairs corresponds to a disease process state of target patients, the witness rate is a percentage of the target patients in the disease process state who are witnessed, the witness action rate is a proportion of witnesses, among all witnesses, of those who take preset rescue measures upon discovering the target patients in the disease process state, and the witnesses belong to a target population;
calculating a rescue action conversion efficiency corresponding to the target organization based on the at least one set of witness information data pairs; wherein the rescue action conversion efficiency is a proportion, among all target patients in the disease process state, of those who receive the preset rescue measures from members of the target population; and
assessing the work performance of the target organization based on the rescue action conversion efficiency corresponding to the target organization.

2. The method according to claim 1, wherein the witness rate is obtained by:
obtaining the witness rate based on a number of the target patients witnessed while in the disease process state and a total number of the target patients in the disease process state.

3. The method according to claim 2, wherein the target patients are out-of-hospital cardiac arrest patients; and
the disease process state is a cardiac arrest onset state, and the obtaining the witness rate based on a number of the target patients witnessed while in the disease process state and a total number of the target patients in the disease process state comprises:
obtaining a first witness rate based on a ratio between a number of the out-of-hospital cardiac arrest patients witnessed while in the cardiac arrest onset state and a total number of the out-of-hospital cardiac arrest patients in the cardiac arrest onset state; or
the disease process state is a post-cardiac arrest unresponsive state, and the obtaining the witness rate based on a number of the target patients witnessed while in the disease process state and a total number of the target patients in the disease process state comprises:
obtaining a second witness rate based on a ratio between a number of the out-of-hospital cardiac arrest patients witnessed while already in the post-cardiac arrest unresponsive state and a total number of the out-of-hospital cardiac arrest patients in the post-cardiac arrest unresponsive state.

4. The method according to claim 1, wherein the witness action rate is obtained by:
obtaining the witness action rate based on a number of the witnesses who take the preset rescue measures when the target patients are in the disease process state and a total number of the witnesses.

5. The method according to claim 4, wherein the target patients are out-of-hospital cardiac arrest patients;
the disease process state is a cardiac arrest onset state, and the obtaining the witness action rate based on a number of the witnesses who take the preset rescue measures when the target patients are in the disease process state and a total number of the witnesses comprises:
obtaining a first witness action rate based on a ratio between a number of the witnesses who take the preset rescue measures when the out-of-hospital cardiac arrest patients are in the cardiac arrest onset state and a total number of the witnesses who witness the out-of-hospital cardiac arrest patients in the cardiac arrest onset state; or
the disease process state is a post-cardiac arrest unresponsive state, and the obtaining the witness action rate based on a number of the witnesses who take the preset rescue measures when the target patients are in the disease process state and a total number of the witnesses comprises:
obtaining a second witness action rate based on a ratio between a number of the witnesses who take preset rescue measures when the out-of-hospital cardiac arrest patients are in the post-cardiac arrest unresponsive state and a total number of the witnesses who witness the out-of-hospital cardiac arrest patients in the post-cardiac arrest unresponsive state; and
the preset rescue measures comprise one or more combinations of: manual cardiopulmonary resuscitation and use of an automated external defibrillator.

6. The method according to claim 1, wherein the calculating a rescue action conversion efficiency corresponding to the target organization based on the at least one set of witness information data pairs comprises:
for each set of witness information data pairs, calculating a product of the witness rate and the witness action rate in the set of witness information data pairs; and
obtaining the rescue action conversion efficiency based on the product of the witness rate and the witness action rate from each set of witness information data pairs.

7. The method according to claim 6, wherein the rescue action conversion efficiency is calculated by the following formula:
$$T = {\sum }_{i = 1}^{n} {k}_{i} {w}_{i} * {t}_{i}$$
where T is the rescue action conversion efficiency, wᵢ is the witness rate in an i-th set of witness information data pairs, tᵢ is the witness action rate in the i-th set of witness information data pairs, n is a number of the at least one set of witness information data pairs, and k; is an effect coefficient corresponding to the i-th set of witness information data pairs, and the effect coefficient is used to represent an effect of being administered the preset rescue measures when the target patients are in the disease process state.

8. The method according to claim 7, wherein the target patients are out-of-hospital cardiac arrest patients;
the at least one set of witness information data pairs comprises: a first witness information data pair and a second witness information data pair, the first witness information data pair corresponds to a cardiac arrest onset state of the out-of-hospital cardiac arrest patients; the second witness information data pair corresponds to a post-cardiac arrest unresponsive state of the out-of-hospital cardiac arrest patients;
the rescue action conversion efficiency corresponding to the target organization is calculated by the following formula:
$$T = {w}_{1} * {t}_{1} + {kw}_{2} * {t}_{2}$$
where Tis the rescue action conversion efficiency corresponding to the target organization, w₁ is the witness rate in the first witness information data pair, t₁ is the witness action rate in the first witness information data pair, w₂ is the witness rate in the second witness information data pair, t₂ is the witness action rate in the second witness information data pair, and k is an effect coefficient determined based on a ratio between a rescue effect when the out-of-hospital cardiac arrest patients are in the post-cardiac arrest unresponsive state and a rescue effect when the out-of-hospital cardiac arrest patients are in the cardiac arrest onset state.

9. The method according to claim 1, wherein the assessing the work performance of the target organization based on the rescue action conversion efficiency corresponding to the target organization comprises:
constructing a rescue action conversion efficiency frequency table based on a plurality of rescue action conversion efficiencies including the rescue action conversion efficiency corresponding to the target organization; wherein each rescue action conversion efficiency in the plurality of rescue action conversion efficiencies corresponds to one organization;
obtaining a percentile corresponding to the target organization based on the rescue action conversion efficiency frequency table; and
obtaining a work performance score for the target organization based on the percentile.

10. A method for assessing work performance of a target organization, comprising:
acquiring, within a business coverage area of the target organization and a preset historical time period, at least one set of trainee witness information data pairs; wherein each set of the trainee witness information data pairs comprises a trainee witness rate and a trainee action rate, and corresponds to a disease process state of target patients; the trainee witness rate is a proportion, among all trainees trained by the target organization, of trainees who witness the target patients in the disease process state; the trainee witness action rate is a proportion, among all the trainees trained by the target organization, of trainees trained by the target organization who take preset rescue measures upon discovering the target patients in the disease process state;
calculating a work performance score of the target organization based on the at least one set of trainee witness information data pairs; and
assessing the work performance of the target organization based on the work performance score of the target organization.

11. The method according to claim 10, wherein the target patients are out-of-hospital cardiac arrest patients;
the disease process state is a cardiac arrest onset state, and the trainee witness rate is obtained by:
obtaining a first trainee witness rate based on a ratio between a number of trainees, among those trained by the target organization, who witness the target patients in the cardiac arrest onset state and a total number of the trainees trained by the target organization; or
the disease process state is a post-cardiac arrest unresponsive state, and the trainee witness rate is obtained by:
obtaining a second trainee witness rate based on a ratio between a number of trainees, among those trained by the target organization, who witness the target patients in the post-cardiac arrest unresponsive state and a total number of the trainees trained by the target organization.

12. The method according to claim 10, wherein the target patients are out-of-hospital cardiac arrest patients;
the disease process state is a cardiac arrest onset state, and the trainee action rate is obtained by:
obtaining a first trainee action rate based on a ratio between a number of the trainees trained by the target organization who take the preset rescue measures upon discovering the target patients in the cardiac arrest onset state and a total number of all the trainees trained by the target organization; or
the disease process state is a post-cardiac arrest unresponsive state, and the trainee action rate is obtained by:
obtaining a second trainee action rate based on a ratio between a number of the trainees trained by the target organization who take the preset rescue measures upon discovering the target patients in the post-cardiac arrest unresponsive state and a total number of all the trainees trained by the target organization; and
the preset rescue measures comprise one or more combinations of: manual cardiopulmonary resuscitation and use of an automated external defibrillator.

13. The method according to claim 10, wherein the calculating a work performance score of the target organization based on the at least one set of trainee witness information data pairs comprises:
for each set of trainee witness information data pairs, calculating a product of the trainee witness rate and the trainee action rate in the set of trainee witness information data pairs; and
obtaining the work performance score of the target organization based on the product of the trainee witness rate and the trainee action rate from each set of trainee witness information data pairs.

14. The method according to claim 13, wherein the target patients are out-of-hospital cardiac arrest patients;
the at least one set of trainee witness information data pairs comprises: a first trainee witness information data pair and a second trainee witness information data pair; the first trainee witness information data pair corresponds to a cardiac arrest onset state; and the second trainee witness information data pair corresponds to a post-cardiac arrest unresponsive state; and
the work performance score of the target organization is calculated by the following formula:
$$F = {a}_{1} * {b}_{1} + {ka}_{2} * {b}_{2}$$
where F is the work performance score of the target organization, a₁ is the trainee witness rate in the first trainee witness information data pair, b₁ is the trainee action rate in the first trainee witness information data pair, a₂ is the trainee witness rate in the second trainee witness information data pair, b₂ is the trainee action rate in the second trainee witness information data pair, and k is an effect coefficient determined based on a ratio between a rescue effect when the out-of-hospital cardiac arrest patients are in the post-cardiac arrest unresponsive state and a rescue effect when the out-of-hospital cardiac arrest patients are in the cardiac arrest onset state.

15. An apparatus for assessing work performance of a target organization, comprising:
a first acquisition module, configured to acquire at least one set of witness information data pairs within a business coverage area of the target organization and within a preset historical time period; wherein each set of the witness information data pairs comprises a witness rate and a witness action rate, and corresponds to a disease process state of target patients; the witness rate is a percentage of the target patients in the disease process state who are witnessed; the witness action rate is a proportion of witnesses, among all witnesses, of those who take preset rescue measures upon discovering the target patients in the disease process state; and the witnesses belong to a target population;
a second acquisition module, configured to calculate a rescue action conversion efficiency corresponding to the target organization based on the at least one set of witness information data pairs; wherein the rescue action conversion efficiency is a proportion, among all target patients in the disease process state, of those who receive the preset rescue measures from members of the target population; and
an assessment module, configured to assess the work performance of the target organization based on the rescue action conversion efficiency corresponding to the target organization.

16. An electronic device, comprising a memory and a processor, wherein the memory stores a computer program, and the processor, when running the computer program, executes the method according to claim 1.

17. A non-transitory computer-readable storage medium, configured to store a computer-readable program, wherein when the computer-readable program is executed, the method according to claim 1 is performed.
